(19) United States
(12) Patent Application Publication
(10) Pub. No.: US 2020/0360575 A1
(43) Pub. Date: Nov. 19, 2020

(54) MEDICAL DEVICE TO AID IN WOUND CARE

(71) Applicant: JUSTIN D. DICKSON LLC, Bellbrook, OH (US)

(72) Inventors: Justin D. Dickson, Bellbrook, OH (US); Christie S. Dickson, Bellbrook, OH (US)

(21) Appl. No.: 16/874,779

(22) Filed: May 15, 2020

Related U.S. Application Data
(60) Provisional application No. 62/849,048, filed on May 16, 2019.

(51) Int. Cl. A61M 1/00; A61F 13/00

(52) U.S. Cl. CPC A61M 1/009; A61M 1/0086; A61F 13/00068

(57) ABSTRACT

A medical device for use in treating wounds using negative pressure wound therapy comprises a base and an isolator. The base is made from a collapsible material and includes an aperture. The isolator is made from a flexible material and includes a central passage that aligns with the aperture of the base when the isolator is coupled to the base. The isolator further includes an effluent passage including a pump aperture for coupling the medical device to an effluent suction pump for removing effluent.

### CROSS REFERENCE TO RELATED APPLICATIONS

This application claims the benefit of U.S. Provisional Patent Application Ser. No. 62/849,048, filed May 15, 2019, entitled “Medical Device to Aid in Wound Care”, the disclosure of which is hereby incorporated by reference.

### BACKGROUND

Various aspects of the present invention relate generally to medical devices and specifically medical devices to aid in wound care using negative pressure.

Negative pressure wound therapy (NPWT) is a method of therapy to promote wound healing by delivering negative pressure at a wound site. NPWT uses a vacuum dressing (i.e., a dressing coupled to a vacuum pump) to promote healing in acute or chronic wounds and enhance healing of second- and third-degree burns. NPWT can also be used to treat pressure ulcers, diabetic ulcers, chronic wounds, or injuries. In NPWT, a dressing (e.g., layers of foam fit to a would site) are applied to the wound site such that some of the dressing is within the wound. A bandage (e.g., a thin film) is applied to the wound site, and a vacuum pump may be controlled continuously or intermittently at a same pressure or different pressures, which helps draw wound edges together, removes infectious materials, and actively promotes formation of granulation tissue.

### BRIEF SUMMARY

According to aspects of the present disclosure, a medical device for use in treating wounds using negative pressure wound therapy comprises a base and an isolator. The base is made from a collapsible material and includes an aperture. The isolator is made from a flexible material and includes a central passage that aligns with the aperture of the base when the isolator is coupled to the base. The isolator further includes an effluent passage including a pump aperture for coupling the medical device to an effluent suction pump for removing effluent.

According to further aspects of the present disclosure, a medical device for use in negative pressure wound therapy comprises a base and an isolator. The base is made from a collapsible, non-permeable material includes an aperture. The isolator is made from a flexible, non-permeable material and includes a central passage that aligns with the aperture of the base when the isolator is coupled to the base. An effluent passage includes a pump aperture that is spaced away from the central passage to allow for an ostomy appliance to be coupled to the central passage for use in negative pressure wound therapy. The pump aperture is for coupling the medical device to an effluent suction pump for removing effluent. Moreover, the effluent passage is a structure that runs a length of the central passage including the effluent reservoir and leads from the central passage of the isolator. The isolator further comprises an effluent reservoir coupled to the effluent passage, and the effluent reservoir collects effluent via ports in the isolator.

According to yet further aspects of the present disclosure, a system for use in negative pressure wound therapy comprises an effluent suction pump and a medical device. The medical device comprises a base and an isolator. The base is made from a collapsible, non-permeable material includes an aperture. The isolator is made from a flexible, non-permeable material and includes a central passage that aligns with the aperture of the base when the isolator is coupled to the base. An effluent passage includes a pump aperture that is spaced away from the central passage to allow for an ostomy appliance to be coupled to the central passage for use in negative pressure wound therapy. The pump aperture is for coupling the medical device to the effluent suction pump for removing effluent. Moreover, the effluent passage is a structure that runs a length of the central passage including the effluent reservoir and leads from the central passage of the isolator. The isolator further comprises an effluent reservoir coupled to the effluent passage, and the effluent reservoir collects effluent via ports in the isolator.

### BRIEF DESCRIPTION OF THE SEVERAL VIEWS OF THE DRAWINGS

FIG. 1 is a drawing of an embodiment of a medical device to aid in wound care using negative pressure wound therapy, according to aspects of the present disclosure;

FIG. 2 is a cross-sectional view of an embodiment of a base of the medical device of FIG. 1, according to various aspects of the present disclosure;

FIG. 3 is a cross-sectional view of an embodiment of an isolator of the medical device of FIG. 1, according to various aspects of the present disclosure; and

FIG. 4 is a cross-sectional view of a suction-line coupler of the medical device of FIG. 1, according to various aspects of the present disclosure.

### DETAILED DESCRIPTION

According to aspects of the present disclosure, a medical device is provided for use in negative pressure wound therapy, where the medical device adapts to a shape of a wound via a collapsible base while an isolator provides structure to the medical device. An effluent channel and effluent suction pump actively remove effluent from the medical device, so there is less buildup of effluent, which helps reduce a chance of contamination of the wound. Embodiments of the medical device can isolate and remove effluent from enteroatmospheric fistulas. When a patient moves, any pooled effluent may end up contaminating the wound. As such, by actively removing the effluent through the effluent passage, there is less chance that a wound will be contaminated by any means—even movement of the patient.

Further, in some embodiments, a liner prevents effluent from the wound from leaking between the base and the isolator, which helps prevent contamination of the wound.

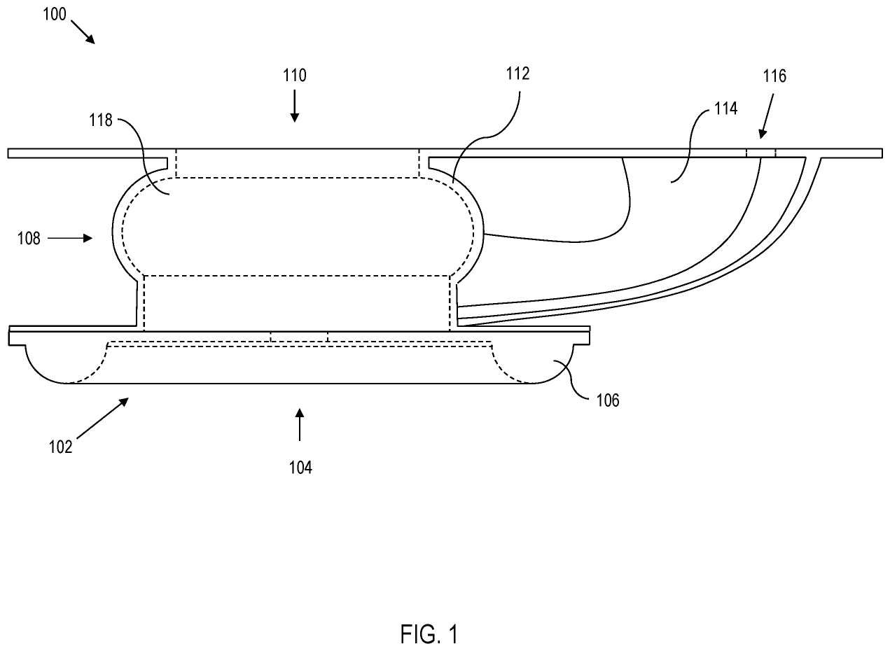

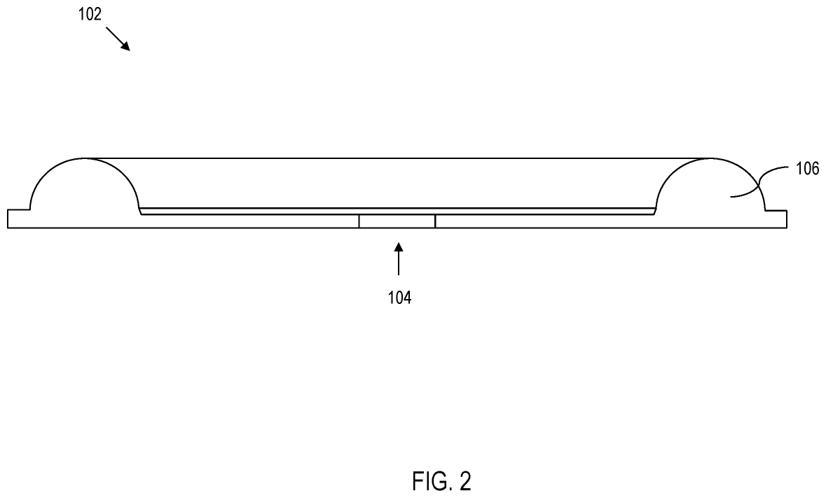

Referring now to the figures, and particularly FIG. 1, a cross sectional view of an embodiment of the medical device 100 is shown. The medical device 100 includes a base 102 of medically safe flexible and/or collapsible material (e.g., gauze, sponge, open-celled foam, nylon, plastic, silicone, etc.). The base includes an aperture 104 near a center of the base 102, and in some embodiments, the base 102 includes a gasket 106 made from similar material as the base 102. As shown, the base 102 is generally circular; however, the base 102 may be any desired shape. The base 102 is discussed in greater detail below in reference to FIG. 2. When a negative pressure is applied to the medical device 100, the base 102 will collapse to a shape of a wound to which the medical device 100 is attached.

The medical device 100 further includes an isolator 108, which may be any flexible material (e.g., plastic, silicone, etc.) and couples to the base 102. The isolator 108 further includes a central passage 110 that is concentric to the aperture 104 of the base when the isolator 108 is coupled to the base 102. In use, a fistula of a patient goes through the aperture 104 and the central passage 110 to receive the negative pressure when the medical device 100 operates.

In some embodiments, a liner 112 maintains a separation between the patient (e.g., the fistula, excretions/effluent from the fistula, etc.) and the rest of the medical device 100. For example, the liner 112 covers an internal portion of the isolator 108 (i.e., a portion of the isolator associated with the central passage 110 of the isolator) and the base 102 continuously. In other words, there are not separate liners between the isolator and the base, because if there were separate liners, then effluent could get between the liners and cause complications at a later time. Thus, the liner 112 isolates the rest of the medical device from possible sources of contamination by covering an interface between the isolator 108 and the base 102. Further, in some embodiments, the liner 112 covers sponge or any other material that may be packed around an outside of the isolator 108. Any material may that can provide a barrier between the medical device 100 and effluent/feculent matter may be used for the liner 112 (e.g., plastic, cellophane, parafilm, etc.).

Moreover, the isolator 108 includes an effluent passage 114 that couples between the isolator 108 and a pump (not shown) via a pump aperture 116. When the pump is operated, a negative pressure is applied to the fistula through the medical device 100 and effluent that is present in the wound is gathered through the effluent passage 114. In various embodiments, the effluent passage 114 is a pelican-bill-shaped, hollow structure that runs a length of the central passage 110 of the isolator 108 and flares out from the central passage 110 (as shown in FIG. 1). In other embodiments, the effluent passage 114 is tubing that extends from a lower portion of the isolator 108 and couples to a pump. Other shapes for the effluent passage 114 are also possible (e.g., a semi-rigid extension from the isolator). Moreover, the effluent passage 114 does not need to extend radially from the isolator 108, but may extend in any direction from the isolator 108. Further, in many embodiments, the liner 112 extends to cover the effluent passage 114 to help mitigate contamination. The pump aperture 116 is spaced away from the from the central channel 110 by a distance that allows for an ostomy appliance (e.g., an ostomy pouch) to be coupled to the isolator 108 over the central channel 110 while the pump aperture 116 is still accessible.

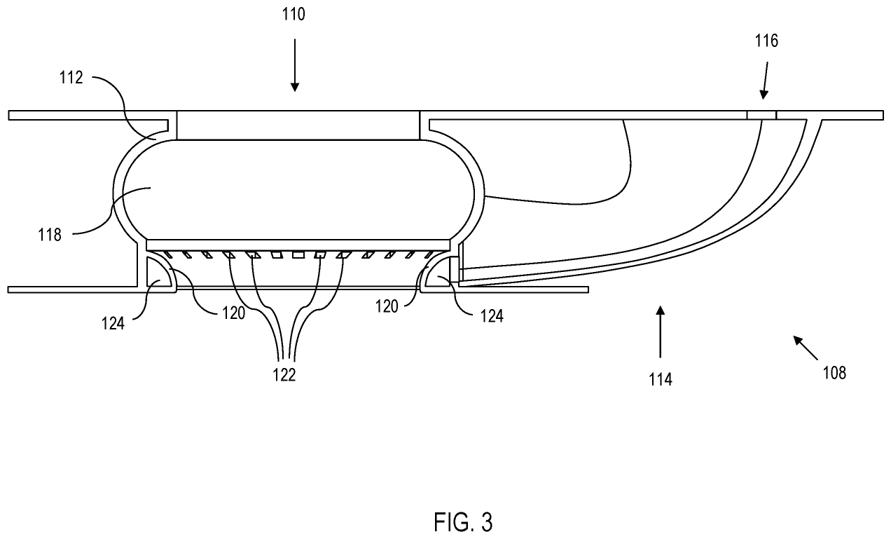

In some embodiments, the central passage 110 is non-uniform through the isolator 108. For example, as shown in FIG. 1, the central passage 110 bulges 118 out to allow for easier compression when negative pressure is applied to the central passage 110. However, other shapes are allowed for the central passage 110 (e.g., cylinder, conical frustum, prism, discrete changes (e.g., cylinder near the bottom into a rectangular prism at the top), multiple bulges 118, etc.). The isolator is discussed further in reference to FIG. 3 below.

As discussed above, the base 102 couples to the isolator 108. However, the coupling may be fixed (i.e., not meant to separate and rejoin) or non-fixed (i.e., removably coupled). If the base 102 and isolator 108 are fixedly coupled, they may be one integral piece or separate pieces that are coupled (e.g., with an adhesive). On the other hand, if the base 102 and isolator 108 are removably coupled, any coupler may be used (e.g., friction fit, screwing the base and isolator, ratchet mechanism, etc.). The pump may be operated continuously, periodically, or intermittently and at a same pressure or different pressures.

Turning now to FIG. 2, a cross section of an embodiment of the base 102 is shown. As discussed above, the base 102 includes an aperture 104 and a gasket 106. As shown, the base 102 is an annulus, but the base 102 may be any desired shape. Further, the base 102 does not need to be the same shape as the aperture 104 or the same shape as the gasket 106. As discussed above, the base 102 may be flexible and/or collapsible material. Moreover, the base 102 may include permeable material, impermeable material, or both. If a permeable material is used, then a liner (as discussed above) should be used to prevent contamination of the material. The gasket 106 is not required; however, including the gasket 106 is preferred to help prevent contamination via effluent.

Further, in some embodiments, the base 102 includes an adhesive opposite the gasket to allow for attachment to devices such as the medical device (100, FIG. 1), an urostomy bag, stoma appliances, etc.

A benefit of the gasket 106 is that it prevents leaks from forming in the base, which eliminates (or reduces) a need for ostomy paste to plug the leaks. Further, the gasket 106 lasts longer than a twenty-four hours that the ostomy paste lasts before the ostomy paste softens and mixes with effluent from the wound. Thus, the gasket 106 creates a less penetrable base 102 than a base without the gasket 106. However, the gasket 106 is not required.

Turning now to FIG. 3, a cross section of an embodiment of the isolator 108 is shown. As described above, the isolator 108 includes an central passage 110 of any desired shape (e.g., the central passage of FIG. 3 includes a bulge 118), a liner 112 that protects from contamination, an effluent passage 114 that allows effluent to be removed from the fistula to which the medical device is attached, and a pump aperture 116 for coupling to an effluent suction pump to provide negative pressure. Further, various embodiments of the isolator 108 include a smoothing section 120 of the central passage 110. The smoothing section 120 provides a continuous (i.e., non-discrete) transition between different portions of the central passage 110. For example, as shown in FIG. 3, the bulge 118 is coupled to a smoothing section 120 to eliminate any edges that may cause damage to the fistula to which the medical device is attached. Moreover, some embodiments of the isolator 108 include ports 122 that lead to an effluent reservoir 124 and allow effluent from the fistula to be gathered in the effluent reservoir 124. Thus, when the effluent suction pump operates, the effluent may be removed from the effluent reservoir 124. As shown in FIG. 3, the effluent reservoir 124 rings around a lower portion of the central channel 110, and the smoothing section 120 forms a wall for the effluent reservoir 124. The ports 122 are in the smoothing section 120 to allow for the effluent to enter the effluent reservoir 124. As shown, the ports 122 are square-shaped, but they can be any desired shape and size (e.g., circular, ovoid, triangular, etc.). Moreover, a spacing between the ports 122 is uniform in FIG. 3, but in some embodiments, the spacing between ports 122 is non-uniform. Further, the spacing may be any distance.

In FIG. 3, the effluent passage 114 is a curved pelican-bill structure that includes a hollow space between panels (as walls). In some embodiments, the pelican-bill effluent passage 114 is a chamber with a V-shaped cross section (as shown in FIG. 3), where the walls are flexible enough to flex outward when the effluent suction pump is activated. Further, a bottom of the pelican-bill effluent passage is curved to aid the effluent as the effluent is being suctioned out of the isolator 108.

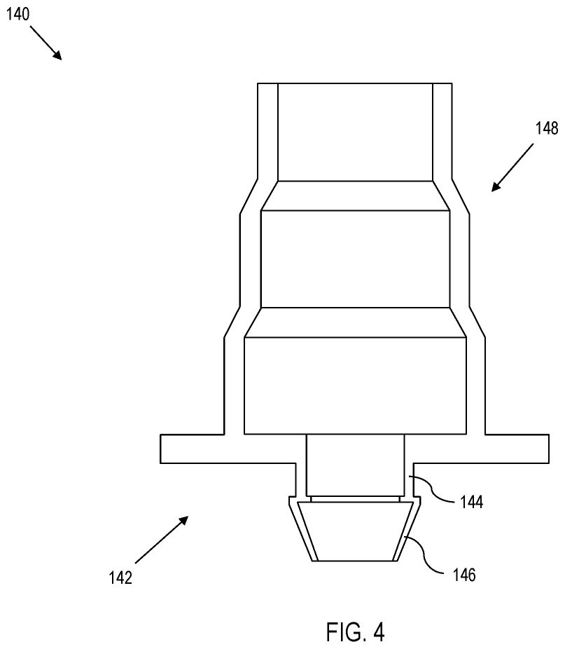

The pump aperture 116 is separated from the central channel 110 by a distance that allows for an ostomy appliance (e.g., an ostomy pouch) to be coupled to the isolator 108 over the central channel 110 while the pump aperture 116 is still accessible. For example, in various embodiments, a center of the pump aperture 116 is eight to nine centimeters from a center of the central channel 110. However, greater distances may be used. Then, the effluent suction pump (which is different than a device that may be used to supply the negative pressure for the wound therapy) may be controlled to remove effluent in the isolator 108. In many embodiments, the effluent suction pump couples to the pump aperture via a suction-line coupler, as described in greater detail in reference to FIG. 4.

Turning now to FIG. 4, a suction-line coupler 140 comprises a protrusion 142 that includes a shaft 144 that is approximately a same size as the pump aperture (116, FIG. 3) in the isolator. Further, a head 146 of the protrusion 142 prevents the protrusion from uncoupling from the isolator when the suction-line coupler 140 is coupled to the isolator. Further, the suction-line coupler 140 comprises a pump coupler 148 that couples to the effluent suction pump.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the invention in the form disclosed. Many modifications and variations will be apparent to those of ordinary skill in the art without departing from the scope and spirit of the invention. Aspects of the disclosure were chosen and described in order to best explain the principles of the invention and the practical application, and to enable others of ordinary skill in the art to understand the invention for various embodiments with various modifications as are suited to the particular use contemplated.

What is claimed is:

1. A medical device for use in treating wounds using negative pressure wound therapy, the device comprising:
a base comprising:
collapsible material; and
an aperture; and
an isolator coupled to the base, the isolator comprising;
flexible material;
a central passage that aligns with the aperture of the base when the isolator is coupled to the base; and
an effluent passage including a pump aperture for coupling the medical device to a pump for removing effluent.

2. The medical device of claim 1, wherein the isolator further comprises:
an effluent reservoir coupled to the effluent passage, wherein the effluent reservoir collects effluent via ports in the isolator.

3. The medical device of claim 2, wherein the effluent passage is a structure that:
runs a length of the central passage including the effluent reservoir; and
leads from the central passage of the isolator.

4. The medical device of claim 3, wherein the pump aperture is spaced away from the central passage to allow for an ostomy appliance to be coupled to the central passage for use in negative pressure wound therapy.

5. The medical device of claim 1, wherein the effluent passage is a structure that:
runs a length of the central passage including the effluent reservoir; and
leads from the central passage of the isolator.

6. The medical device of claim 1, wherein the base includes a gasket that interfaces with a wound.

7. The medical device of claim 1 further comprising:
material to be packed in a wound to which the medical device is coupled, wherein the material is non-permeable.

8. The medical device of claim 1 further comprising:
permeable material to be packed in a wound to which the medical device is coupled, wherein the liner covers the permeable material.

9. The medical device of claim 1, wherein the effluent passage is tubing leading from the central passage of the isolator.

10. The medical device of claim 9, wherein the effluent passage is a pelican-bill-type structure.

11. The medical device of claim 1, wherein the central passage of the isolator is non-uniform.

12. The medical device of claim 11, wherein the central passage of the isolator includes a bulge.

13. The medical device of claim 11, wherein the central passage of the isolator includes multiple bulges.

14. The medical device of claim 11, wherein the central passage of the isolator is a conical frustum.

15. The medical device of claim 11, wherein the central passage of the isolator is continuous between transitions in size.

16. The medical device of claim 1, wherein the central passage of the isolator is a cylinder.

17. The medical device of claim 1, wherein:
the base and the isolator are two separate pieces; and
a liner covers an interface between the base and the isolator.

18. A medical device for use in negative pressure wound therapy, the medical device comprising:
a base comprising:
collapsible, non-permeable material; and
an aperture; and
an isolator coupled to the base, the isolator comprising;
flexible non-permeable material;
a central passage that aligns with the aperture of the base when the isolator is coupled to the base;
an effluent passage including a pump aperture that is spaced away from the central passage to allow for an ostomy appliance to be coupled to the central passage for use in negative pressure wound therapy, wherein the pump aperture is for coupling the medical device to a pump for removing effluent, wherein the effluent passage is a structure that:
runs a length of the central passage including the effluent reservoir; and
leads from the central passage of the isolator; and
an effluent reservoir coupled to the effluent passage, wherein the effluent reservoir collects effluent via ports in the isolator.

19. A system for use in negative pressure wound therapy, the system comprising:
an effluent suction pump; and
a medical device comprising:
a base comprising:
collapsible, non-permeable material; and
an aperture; and
an isolator coupled to the base, the isolator comprising;
flexible non-permeable material;
a central passage that aligns with the aperture of the base when the isolator is coupled to the base;
an effluent passage including a pump aperture that is spaced away from the central passage to allow for an ostomy appliance to be coupled to the central passage for use in negative pressure wound therapy, wherein the pump aperture is for coupling the medical device to the effluent suction pump for removing effluent, wherein the effluent passage is a structure that:
runs a length of the central passage including the effluent reservoir; and
leads from the central passage of the isolator; and
an effluent reservoir coupled to the effluent passage, wherein the effluent reservoir collects effluent via ports in the isolator.
